(19) United States
(12) Patent Application Publication
(10) Pub. No.: US 2002/0110558 A1
(43) Pub. Date: Aug. 15, 2002

(54) Use of CD25 binding molecules in the treatment of rheumatoid arthritis or skin diseases

(76) Inventors: Peter Lloyd Amlot, London (GB); Max H. Schreier, Basel (CH); Karin Schreier, Basel (CH)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 09/770,002

(22) Filed: Jan. 25, 2001

Related U.S. Application Data
(63) Continuation of application No. PCT/EP99/05316, filed on Jul. 26, 1999.

(30) Foreign Application Priority Data
Jul. 27, 1998 (GB) 9816281.1
May 27, 1999 (GB) 9912460.4

(51) Int. Cl. A61K 39/395; C07K 16/28

(52) U.S. Cl. 424/143.1; 530/388.22

(57) ABSTRACT

Use of a CD25 binding molecule which comprises at least one antigen binding site comprising at least one domain which comprises in sequence, the hypervariable regions CDR1, CDR2 and CDR3; said CDR1 having the amino acid sequence Arg-Tyr-Trp-Met-His, said CDR2 having the amino acid sequence Ala-Ile-Tyr-Pro-Gly-Asn-Ser-Asp-Thr-Ser-Tyr-Asn-Gln-Lys-Phe-Glu-Gly, and said CDR3 having the amino acid sequence Asp-Tyr-Gly-Tyr-Tyr-Phe-Asp-Phe, in the treatment of rheumatoid arthritis or inflammatory or hyperproliferative skin diseases.

The invention is directed to the use of a CD25 binding molecule in the treatment of rheumatoid arthritis or inflammatory and hyperproliferative skin diseases.

More specifically the present invention provides in a first aspect the use of a CD25 binding molecule which comprises at least one antigen binding site comprising at least one domain which comprises in sequence, the hypervariable regions CDR1, CDR2 and CDR3; said CDR1 having the amino acid sequence Arg-Tyr-Trp-Met-His, said CDR2 having the amino acid sequence Ala-lle-Tyr-Pro-Gly-Asn-Ser-Asp-Thr-Ser-Tyr-Asn-Gln-Lys-Phe-Glu-Gly, and said CDR3 having the amino acid sequence Asp-Tyr-Gly-Tyr-Tyr-Phe-Asp-Phe; or direct equivalents thereof in the treatment of rheumatoid arthritis or inflammatory or hyperproliferative skin diseases.

Treatment of rheumatoid arthritis includes control or amelioration of the disease and/or its sequellae, e.g. symptoms, such as number of tender and swollen joints, degree of tenderness and swelling, or pain, as well as control or amelioration of aetiological components.

Treatment of inflammatory and hyperproliferative skin disease includes control or amelioration of the disease and/or its sequellae, e.g. symptoms, as well as control or amelioration of aetiological components. Treatment of e.g. psoriasis also includes suppression of further clinical relapse.

Inflammatory and hyperproliferative skin diseases include psoriasis, atopical dermatitis, contact dermatitis and further eczematous dermatitises, seborrhoeic dermatitis, Lichen planus, Pemphigus, bullous Pemphigoid, Epidermolysis bullosa, urticaria, angioedemas, vasculitides, erythemas, cutaneous eosinophilias, Lupus erythematosus and acne.

By “CD25 binding molecule” is meant any molecule capable of binding to the CD25 antigen either alone or associated with other molecules to form high affinity IL-2receptors;

Preferably a CD25 binding molecule is used comprising at least one antigen binding site comprising:

a) a first domain comprising in sequence the hypervariable regions CDR1, CDR2 and CDR3; said CDR1 having the amino acid sequence Arg-Tyr-Trp-Met-His, said CDR2 having the amino acid sequence Ala-lle-Tyr-Pro-Gly-Asn-Ser-Asp-Thr-Ser-Tyr-Asn-Gln-Lys-Phe-glu -Gly, and said CDR3 having the amino acid sequence Asp-Tyr-Gly-Tyr-Tyr-Phe-Asp-Phe and,

b) a second domain comprising in sequence the hypervariable regions CDR1′, CDR2′ and CDR3′, said CDR1′ having the amino acid sequence Ser-Ala-Ser-Ser-Ser-lle-Ser-Tyr-Met-Gin, said CDR2′ having the amino acid sequence Asp-Thr-Ser-Lys-Leu-Ala-Ser, and said CDR3′ having the amino acid sequence His-ln-Arg-Ser-Ser-Tyr-Thr; or direct equivalents thereof.

Unless otherwise indicated, any polypeptide chain is herein described as having an amino acid sequence starting at the N-terminal extremity and ending at the C-terminal extremity.

When the antigen binding site comprises both the first and second domains, these may be located on the same polypeptide molecule or, preferably, each domain may be on a different chain, the first domain being part of an immunoglobulin heavy chain or fragment thereof and the second domain being part of an immunoglobulin light chain or fragment thereof.

Accordingly, the invention also provides the use of a CD25 binding molecule which comprises at least one antigen binding site comprising either a first domain having an amino acid sequence identical or substantially identical to that shown in Seq. Id. No. 1 in EP 449,769, the contents of which is herein incorporated by reference, starting with amino acid at position 1 and ending with amino acid at position 117 or a first domain as described above and a second domain having an amino acid sequence identical or substantially identical to that shown in Seq. Id. No. 2 in EP 449,769, the contents of which is herein incorporated by reference, starting with amino acid at position 1 and ending with amino acid at position 104 in the treatment of rheumatoid arthritis or inflammatory and hyperproliferative skin diseases.

A more preferred CD25 binding molecule for use in accordance with the invention is selected from a chimeric anti-CD25 antibody which comprises at least

a) one immunoglobulin heavy chain or fragment thereof which comprises (i) a variable domain comprising in sequence the hypervariable regions CODR1, CDR2 and CDR3 and (ii) the constant part or fragment thereof of a human heavy chain; said CDR1 having the amino acid sequence Arg-Tyr-Trp-Met-His, said CDR2 having the amino acid sequence Ala-Ile-Tyr-Pro -Gly-Asn-Ser-Asp-Thr-Ser-Tyr-Asn-Gln-Lys-Phe-Glu-Gly, and said CDR3 having the amino acid sequence Asp-Tyr-Gly-Tyr-Tyr-Phe-Asp-Phe and

b) one immunoglobulin light chain or fragment thereof which comprises (i) a variable domain comprising in sequence the hypervariable regions CDR1′, CDR2′ and CDR3′ and (ii) the constant part or fragment thereof of a human light chain; said CDR1′ having the amino acid sequence Ser-Ala-Ser-Ser-Ser-lle-Ser-Tyr-Met-Gln, said CDR2′ having the amino acid sequence Asp-Thr-Ser-Lys-Leu-Ala-Ser, and said CDR3′ having the amino acid sequence His-Gln -Arg-Ser-Ser-Tyr-Thr, and direct equivalents thereof.

Alternatively, a CD25 binding molecule for use in accordance with the invention may be selected from a single chain binding molecule which comprises an antigen binding site comprising

a) a first domain comprising in sequence the hypervariable regions CDR1, CDR2 and CDR3, said hypervariable regions having the amino acid sequences as shown in Seq. Id. No. 1 in EP 449,769, the contents of which is herein incorporated by reference,

b) a second domain comprising in sequence the hypervariable regions CDR1′, CDR2′ and CDR3′, said hypervariable regions having the amino acid sequences as shown in Seq. Id. No. 2 in EP 449,769, the contents of which is herein incorporated by reference, and

c) a peptide linker which is bound either to the N-terminal extremity of the first domain and to the C-terminal extremity of the second domain or to the C-terminal extremity of the first domain and to the N-terminal extremity of second domain; and direct equivalents thereof.

As it is well known, minor changes in an amino acid sequence such as deletion, addition or substitution of one or several amino acids may lead to an allelic form of the original protein which has identical or substantially identical properties. Thus, by the term “direct equivalents thereof” is meant either any single domain CD25 binding molecule (molecule X)

(i) in which the hypervariable regions CDR1, CDR2 and CDR3 taken as a whole are at least 80% homologous, preferably at least 90% homologous, more preferably at least 95% homologous to the hypervariable regions as shown in Seq. Id. No. 1 in EP 449,769, the contents of which is herein incorporated by reference, and,

(ii) which is capable of inhibiting the binding of Interleukin 2 (IL-2) to its receptor substantially to the same extent as a reference molecule having framework regions identical to those of molecule X but having hypervariable regions CDR1, CDR2 and CDR3 identical to those shown in Seq. Id. No. 1 in EP 449,769, the contents of which is herein incorporated by reference;

or any CD25 binding molecule having at least two domains per binding site (molecule X′)

(i) in which the hypervariable regions CDR1, CDR2, CDR3, CDR1′, CDR2′ and CDR3′ taken as a whole are at least 80% homologous, preferably at least 90% homologous, more preferably at least 95% homologous to the hypervariable regions as shown in Seq. Id. No. 1 and 2 in EP 449,769, the contents of which is herein incorporated by reference, and

(ii) which is capable of inhibiting the binding of IL-2 to its receptor substantially to the same extent as a reference molecule having framework regions and constant parts identical to molecule X′ but having hypervariable regions CDR1, CDR2, CDR3, CDR1′, CDR2′ and CDR3′ identical to those shown in Seq. Id. No. 1 and 2 in EP 449,769, the contents of which is herein incorporated by reference.

This last criterion may be conveniently tested in various assays as described in EP 449,769, the contents of which is herein incorporated by reference.

A most preferred CD25 binding molecule for use in accordance with the invention is a chimeric CD25 antibody, especially a chimeric CD25 antibody comprising at least

a) one heavy chain which comprises a variable domain having an amino acid sequence identical or substantially identical to that shown in Seq. Id. No. 1 in EP 449,769, the contents of which is herein incorporated by reference, starting with amino acid at position 1 and ending with amino acid at position 117 and the constant part of a human heavy chain; and

b) one light chain which comprises a variable domain having an amino acid sequence identical or substantially identical to that shown in Seq. Id. No. 2 in EP 449,769, the contents of which is herein incorporated by reference, starting with glutamic acid at position 1 and ending with glutamic acid at position 104 and the constant part of a human light chain.

The constant part of a human heavy chain may be of the γ1, γ2, γ3, γ4, μ, α1, α2, δ or ε type, preferably of the γ type, more preferably of the γ1 type, whereas the constant part of a human light chain may be of the κ or λ type (which includes the λ1, λ2 and λ3 subtypes) but is preferably of the κ type. The amino acid sequence of all these constant parts are given in Kabat et al., Sequences of Proteins of Immunological Interest, US Department of Health and Human Services, Public Health Service, NIH.

The most preferred CD25 binding molecule is basiliximab which is commercially available as SIMULECT® from Novartis AG.

The CD25 binding molecules suitable for use in accordance with the present invention may be produced by techniques disclosed for example in EP 449,769, the contents of which is herein incorporated by reference, in particular in Examples 1 to 5 of EP 449,769.

As described in EP-B-449,769, the CD25 binding molecules have, on the basis of observed activity in e.g. a Mixed Lymphocyte Reaction assay, been found to be useful for preventing or treating graft rejection episodes.

In accordance with the present invention it has now surprisingly been found that the CD25 binding molecules are effective in the treatment of rheumatoid arthritis or inflammatory or hyperproliferative skin diseases.

Therefore the invention also provides

(i) A method for the treatment of rheumatoid arthritis or inflammatory or hyperproliferative skin diseases in a patient in need of such treatment comprising administering to the patient an effective amount of a CD25 binding molecule as described above.

(ii) A method for the treatment of rheumatoid arthritis or inflammatory or hyperproliferative skin diseases in a subject in need of such treatment comprising administrating, e.g. concomitantly or in sequence, to said subject an effective amount of a) a CD25 binding molecule as described above and b) a further drug substance being effective in the treatment of rheumatoid arthritis or inflammatory or hyperproliferative skin diseases.

(iii) A method for the treatment of inflammatory or hyperproliferative skin diseases in a subject in need of such treatment comprising administrating an effective amount of a CD25 binding molecule as described above and applying a non-drug inflammatory or hyperproliferative skin disease therapy, e.g. UV light therapy, to said subject.

(iv) A method as described in (ii) combined with a method as described in (iii).

(v) A pharmaceutical composition for use in any of the methods as described in (i) to (iv) which comprises a CD25 binding molecule as described above and a pharmaceutically acceptable carrier or diluent.

(vi) A CD25 binding molecule as described above for use in the manufacturing of a medicament for use in any of the methods as described in (i) to (iv).

(vii) A therapeutic combination, e.g. a kit or package, for use in any of the methods as described in (i) to (iv) said combination including a pharmaceutical composition comprising a CD25 binding molecule as described above, and further including at least one pharmaceutical composition comprising a further drug substance effective in the treatment of rheumatoid arthritis or inflammatory or hyperproliferative skin diseases.

For the use in accordance with the invention, the appropriate dosage will, of course, vary depending upon, for example, the particular CD25 binding molecule to be employed, the host, the mode of administration and the severity of the condition being treated and the effects desired. Satisfactory results are generally indicated to be obtained at dosages from about 0.1 mg to about 100 mg. Administration may be in a single dose or in several doses over a period of time as long as may be indicated in relation to the time the disease is clinically evident or prophylactically to suppress further clinical relapse, for example a dose of 10 to 100 mg may be administered with a time-lag of one week to five weeks, e.g. every four weeks, up to a time control or amelioration of the disease is achieved. The CD25 binding molecule is conveniently administered parenterally, e.g. intravenously, for example, into the antecubital or other peripheral vein. An exemplary dosing regimen is intravenous administration of 40 mg every 28 days until control or amelioration of the disease is achieved.

Pharmaceutical compositions of the invention may be manufactured in a conventional manner as described, e.g. in EP 449,769, the contents of which is herein incorporated by reference.

The CD25 binding molecule may be administered as the sole active ingredient or together with other drugs in immunomodulating regimens or other anti-inflammatory agents. For example, the CD25 binding molecule may be used in accordance with the invention in combination with cyclosporins, rapamycins or ascomycins, or their immunosuppressive analogs, e.g. cyclosporin A, cyclosporin G, FK-506, rapamycin etc.; corticosteroids e.g. prednisone; cyclophosphamide; azathioprene; methotrexate; gold salts, sulfasalazine, antimalarials, brequinar; leflunomide; mizoribine; mycophenolic acid; mycophenolate mofetil; 15-deoxyspergualine; other immuno-suppressive monoclonal antibodies, e.g. monoclonal antibodies to leukocyte receptors, e.g. MHC, CD2, CD3, CD4, CD7, CD28, B7, CD40, CD45, or CD58 or their ligands; or other immunomodulatory compounds, e.g. CTLA4lg.

If the CD25 binding molecule is co-administered with a further drug substance both may be packaged separately within the same container, with instructions for mixing or concomitant administration. Examples of kits include for example a multi-barrelled syringe or a twin pack containing separate unit dose forms.

The intravenous infusions may be prepared as follows: the lyophylized antibodies are mixed together and dispersed into 100 ml sterile buffered saline containing 4.5% wt. of human albumin. This saline dispersion may be administered to the patients either as an intravenous bolus injection or as an intravenous infusion over a 15 minute period.

Investigations so far indicate that the administration of the CD25 binding molecules is free from unacceptable side-effects at the dosage levels employed. Particularly the preferred one, basiliximab, is safe, approved by the Federal Drug Administration (FDA) of the United States and is commercially available.

Utility of the CD25 binding molecule for the treatment of rheumatoid arthritis is shown in the following clinical examples.

60 patients with active rheumatoid arthritis and having a partial response to methotrexate alone are enrolled. A partial response to methotrexate alone is defined as the presence of at least 6 swollen and 9 tender joints and C-reactive protein level >20 mg/l in patients who have been receiving a stable, maximally tolerated methotrexate dose (not exceeding 20 mgl/week) for at least 3 months prior to the screening visit (Week -2).

These 60 patients are randomized to one of two treatment groups; those receiving methotrexate plus basiliximab and those receiving methotrexate plus placebo. The methotrexate dose and route of administration remains stable during the course of the study. The patients are also on stable doses of nonsteroidal anti-inflammatory drugs and-prednisone.

### EXAMPLE 1

Basiliximab is administered intravenously at a dose of 60 mg on Day 0, 40 mg on Day 28 and 40 mg on Day 56. Patients are evaluated at Weeks 2, 4, 6, 8, 10 and 12 for safety, efficacy and disease outcome.

The primary efficacy outcome measure is the attainment of ACR (American College of Rheumatology) criteria for improvement of rheumatoid arthritis at Week 12. ACR (20) criteria defines improvement as 20% improvement in the number of tender and swollen joints, in addition to 20% improvement in at least three of five variables (degree of disability, HAQ (Health Activity Questionnaire); patient global assessment; physician global assessment; pain and C-reactive protein).

Patients receiving basiliximab show an amelioration of the symptoms as compared to patients receiving placebo.

### EXAMPLE 2

Basiliximab is administered intravenously at a dose of 0.02 mg on Day 0, 0.2 mg on Day 4, 2 mg on Day 8, 20 mg on Day 12, 40 mg on Day 19 and 60 mg on Day 26. Patients are evaluated at Weeks 2, 4, 6, 8, 10 and 12 for safety, efficacy and disease outcome as in Example 1.

Patients receiving basiliximab show an amelioration of the symptoms as compared to patients receiving placebo.

Utility of the CD25 binding molecules in the treatment of inflammatory or hyperproliferative skin diseases, e.g. psoriasis, is shown in the following clinical example.

24 patients (both male and female) between the ages of 18-70 years of age with moderate to severe plaque psoriasis defined as having a regional or total PASI≧10 and affected BSA≧5% enter into the Screening period (Visit 1, Week -2) and are randomized into one of two CD25 binding molecule dose groups (40 mg or 60 mg).

A CD25 binding molecule, e.g. basiliximab is administered at 40 mg or 60 mg (depending on initial randomization), all subsequent 40 mg or 60 mg 15 min iv infusion doses being administered within 48 h of receipt of every blood flow cytometry measurement demonstrating unsaturated peripheral blood lymphocyte IL-2 receptors during a 12 week treatment period. Blood draws for blood flow cytometry measurement occur every 2-weeks at each scheduled patient visit to measure the percent peripheral blood lymphocyte IL-2 receptor saturation. Saturation is defined and reported as a ≧95% reduction in CD25 cells with complete IL-2 receptor saturation and unsaturation is defined and reported as ≦95% CD25 cells with complete IL-2 receptor saturation.

Each patient is evaluated every 2 weeks for efficacy (PASI, % affected BSA, global and overall psoriasis evaluation) and photographed using standard photographic methods. Calculation of the PASI: The head (0.1), trunk (0.3), upper limbs (0.2) and lower limbs (0.4) are assessed separately for erythema, infiltration, scaling. The average degree of severity of each symptom in each of the four body parts, is assigned a score of 0-4. The area covered by lesions on each body part is estimated as a percentage of the total area of that particular body part and score from 0-6 is then assigned. This evaluation takes into account the true area affected and not a ‘functional’ area affected. Further practical details help the assessment: The buttocks count as part of the legs; the axillae and groin count as part of the trunk; the neck counts as part of the head; when scoring the severity of erythema, scales are not removed.

PASI - Scoring System

| Score | 0 | 1 | 2 | 3 | 4 |
|---|---|---|---|---|---|
| Erythema | none | slight | moderate | severe | very |
| Infiltration | | | | | severe |
| Desquamation | | | | | |

PASI - Scoring System

| Score | 0 | 1 | 2 | 3 | 4 | 5 | 6 |
|---|---|---|---|---|---|---|---|
| True | 0% | 1%-9% | 10%-29% | 30%-49% | 50%-69% | 70%-89% | 90%-100% |
| Area % | | | | | | | |

The total score for each body part is obtained by multiplying the sum of the severity scores of the 3 basic lesions by the area score, then multiplying the result by the constant weighted value (head=0.1, trunk=0.3, upper limbs=0.2, lower limbs=0.4) assigned to that body part. The sum of the scores of the individual body parts gives the PASI.

Affected surface determination (rule of 9's): An estimate of the percent BSA affected with psoriasis is estimated. Each section of the body is labeled with the percent of the total BSA it represents.

Overall psoriasis evaluation: An overall evaluation by patient and investigator is performed comparing impression of improvement while on the drug when related to pretreatment condition. The following scale is used: 1=very good; 2=good; 3=moderate; 4=slight; 5=none.

The investigator's and patient's overall psoriasis evaluation are performed at Visit 6 (Week 6), Visit 7 (Week 8), Visit 8 (Week 10), Visit 9 (Week 12), Visit 10 (Week 16), and Visit 11 (Week 20).

Global psoriasis evaluation: A global evaluation of the current status of the disease is rated by the investigator using the following scale: 1=totally clear; 2=almost clear; 3=mild; 4=mild-moderate; 5=moderate; 6=moderate-severe; 7=severe.

Patients receiving basiliximab show a marked improvement with respect to PASI, Global Psoriasis Evaluation, BSA, and Overall Psoriasis Evaluation as compared to patients receiving placebo.

1. A CD25 binding molecule which comprises at least one antigen binding site comprising at least one domain which comprises in sequence, the hypervariable regions CDR1, CDR2 and CDR3; said CDR1 having the amino acid sequence Arg-Tyr-Trp-Met-His, said CDR2 having the amino acid sequence Ala-lle-Tyr-Pro-Gly-Asn-Ser-Asp-Thr-Ser-Tyr-Asn-Gln-Lys-Phe -Glu-Gly, and said CDR3 having the amino acid sequence Asp-Tyr-Gly-Tyr-Tyr-Phe-Asp -Phe; or direct equivalents thereof for use in the treatment of rheumatoid arthritis or inflammatory or hyperproliferative skin diseases.

2. A CD25 binding molecule according to claim 1 for use in the manufacturing of a medicament for use in the treatment of rheumatoid arthritis or inflammatory or hyperproliferative skin diseases in a patient in need of such treatment comprising administering to the patient an effective amount of a CD25 binding molecule according to claim 1.

3. A pharmaceutical composition for the treatment of rheumatoid arthritis or inflammatory or hyperproliferative skin diseases comprising a CD25 binding molecule according to claim 1 and a pharmaceutically acceptable carrier or diluent.

4. A method for the treatment of rheumatoid arthritis or inflammatory or hyperproliferative skin diseases in a patient in need of such treatment comprising administering to the patient an effective amount of a CD25 binding molecule according to claim 1.

5. A method for the treatment of rheumatoid arthritis or inflammatory or hyperproliferative skin diseases in a subject in need of such treatment comprising administrating to said subject an effective amount of a) a CD25 binding molecule according to claim 1 and b) a further drug substance being effective in the treatment of rheumatoid arthritis or inflammatory or hyperproliferative skin diseases.

6. A method for the treatment of inflammatory or hyperproliferative skin diseases in a subject in need of such treatment comprising administrating an effective amount of a CD25 binding molecule according to claim 1 and applying a non-drug inflammatory or hyperproliferative skin disease therapy, e.g. UV light therapy, to said subject.

7. A therapeutic combination for use in any of the methods as described in (i) to (iv) said combination including a pharmaceutical composition comprising a CD25 binding molecule according to claim 1, and further including at least one pharmaceutical composition comprising a further drug substance effective in the treatment of rheumatoid arthritis or inflammatory or hyperproliferative skin diseases.

8. A method according to any one of claims 4 to 6, wherein the CD25 binding molecule is basiliximab.

9. A CD25 binding molecule according to claim 1 or 2, which is basiliximab.

10. A composition or combination according to claim 3 or 7, wherein the CD25 binding molecule is basiliximab.
